(19) United States
(12) Patent Application Publication
(10) Pub. No.: US 2023/0124131 A1
(43) Pub. Date: Apr. 20, 2023

(54) VENTILATION MANIFOLD AND SYSTEM

(71) Applicant: JFD LTD, Glasgow (GB)

(72) Inventors: Callum CRAWFORD, Westhill (GB); Peter RAFFERTY, Westhill (GB); Stephen BLATHERWICK, Westhill (GB); Gareth KERR, Westhill (GB); Danny GRAY, Westhill (GB)

(21) Appl. No.: 17/914,217

(22) PCT Filed: Mar. 24, 2021
(86) PCT No.: PCT/IB2021/052448

§ 371 Date: Sep. 23, 2022

(30) Foreign Application Priority Data
Mar. 24, 2020 (GB) 2004281.8

(51) Int. Cl. A61M 16/08; A61M 16/20; A61M 16/06; A61M 16/12

(52) U.S. Cl. CPC A61M 16/0833; A61M 16/208; A61M 16/06; A61M 16/122; A61M 2202/0208

(57) ABSTRACT

An aspect provides a ventilation manifold for a ventilator, the manifold comprising a fluid flow path, the flow path comprising: bifurcated end paths, a constriction path and a compressed breathable gas chamber fluidly coupled to a compressed breathable gas source, the manifold having a manifold axis defined by the constriction path; a source path of the bifurcated ends coupled to an outlet conduit for a ventilator to present the compressed breathable gas at a source angle to one side of the manifold axis; and a vent path at a vent angle to the manifold axis for an exhaust flow, the vent path acting to provide a fluid pressure valve upon an inlet compressed breathable gas from the constriction path to urge flow towards the source path and a by-pass for returned outlet spent gas flow from the source path against the inlet compressed breathable gas at the constriction path, the fluid pressure valve and the by-pass dependent upon the relative magnitudes of the source angle and the vent angle to the manifold axis and/or each other along with the configuration of the constriction path and/or the configuration of the source path and/or the vent path.

This specification relates to patient ventilators and ventilation systems. In particular, although not exclusively, this specification relates to multi-place BiPAP non-invasive patient ventilation systems for pre-critical care applications where patient breathing assistance is required. Further, it is a non-exclusive object of this specification to provide a patient ventilation system which may allow the adjustment and pre-setting of key treatment parameters that will allow use by a range of patients.

Ventilation systems may be used by a patient to assist breathing where the patient is experiencing respiratory difficulties.

One form of ventilation is non-invasive ventilation (NIV) which relies upon the patient wearing a face mask or similar that allows ventilation without invasive intubation and therefore can be administered to patients who are able to maintain an airway and some breathing function but who require assistance.

Such ventilation techniques use an increased positive pressure of breathing gas to assist patients during recovery from respiratory failure (RF).

The use of such ventilation is useful in supporting patients while the underlying cause or condition that has led to the RF is reversed. For patients who are heading into RF this technique can provide assistance and breathing support for a critical period, for example, while a patient's immune system deals with an underlying viral infection. Importantly, the use of NIV may prevent declining patient health that would normally result in the need for invasive ventilation.

There are a number of different techniques used in NIV therapy, some more complex than others. Two known techniques are CPAP and BiPAP.

The BiPAP technique refers to Bi-level Positive Airway Pressure where a different pressure is supplied to the patient during the inspiration and expiration phases of the breathing cycle. This technique is suited to the treatment of both Type I and Type II hypercapnic RF or a combination of both. For this reason, the use of BiPAP is generally considered as more effective and versatile than simple CPAP as it allows actual breathing assistance during a complete breathing cycle.

For conventional mechanical non-invasive clinical ventilators, BiPAP (or a derivative thereof) may be the preferred mode of operation/treatment, and the critical breathing parameters of pressure, flow and volume (over time) are adjustable by clinicians.

The design and manufacture of fully adjustable BiPAP ventilator systems is complex and involved, particularly where parameters are variable over a wide operating range and a wide range of patients. There are also issues with electrical power consumption and use of compressed oxygen gas supplies which may be considerable where a large number of ventilator systems are rapidly deployed.

There is, therefore, a need to provide a ventilation system which alleviates one or more problems associated with the prior art.

Accordingly, a first aspect is provided by a ventilator manifold as claimed in claim 1. Further aspects of the manifold are provided in dependent claims 2 to 18.

In accordance with a second aspect of the present invention, there is provided a ventilator system as claimed in claim 29. Further aspects of the system are provided by dependent claims 30 to 38.

Embodiments of the ventilation manifold and system are described, by way of example, with reference to the accompanying drawings, in which:

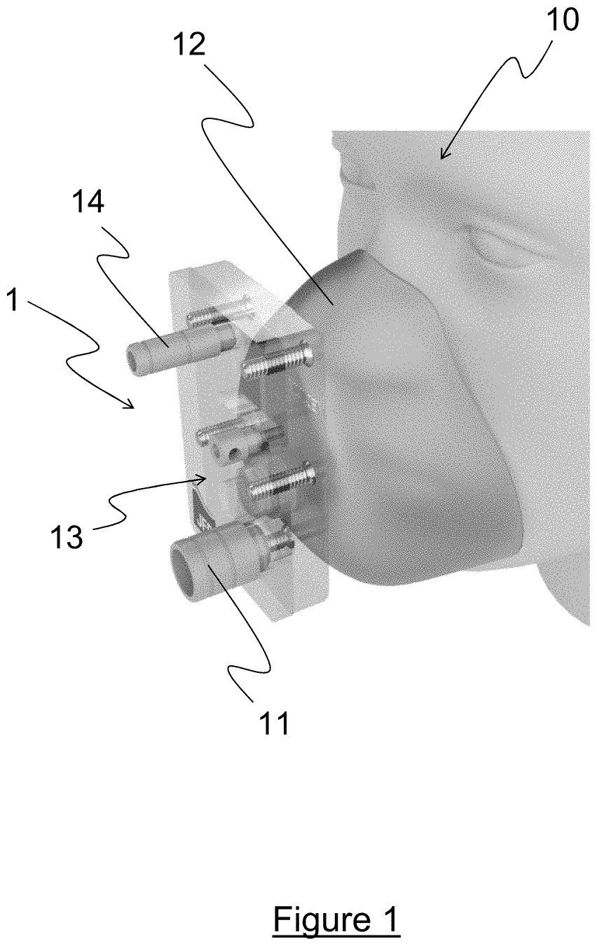

FIG. 1 show the typical size and mounting arrangement of a ventilator on a patient;

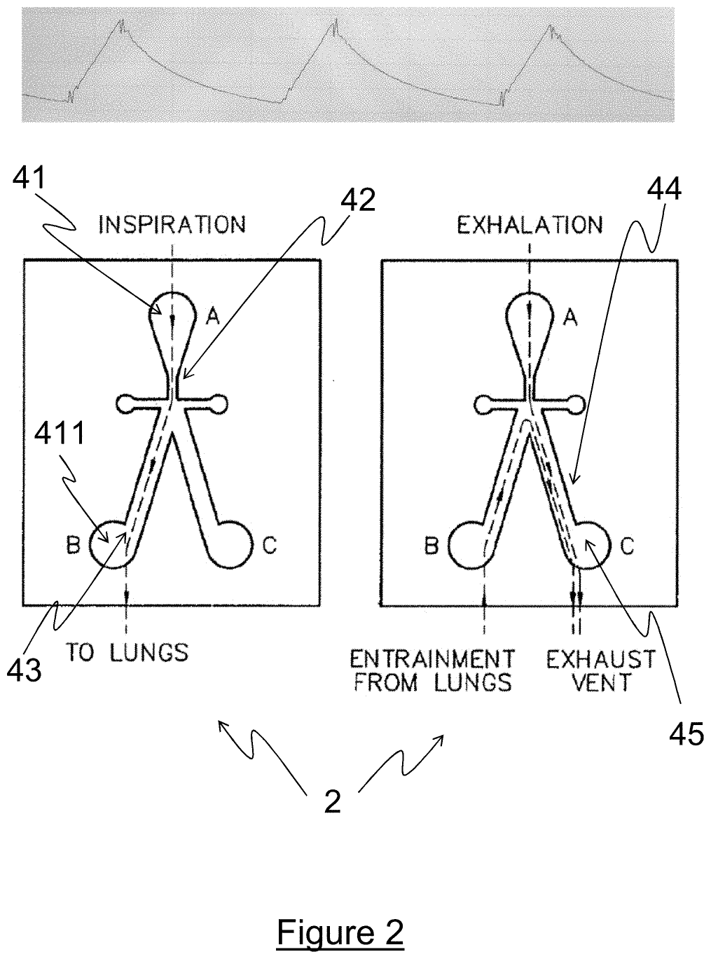

FIG. 2 shows a typical breathing cycle graph (y-axis barometric pressure, x-axis time) and a cross-section of a logic element showing gas flow during inspiration and exhalation breathing phases;

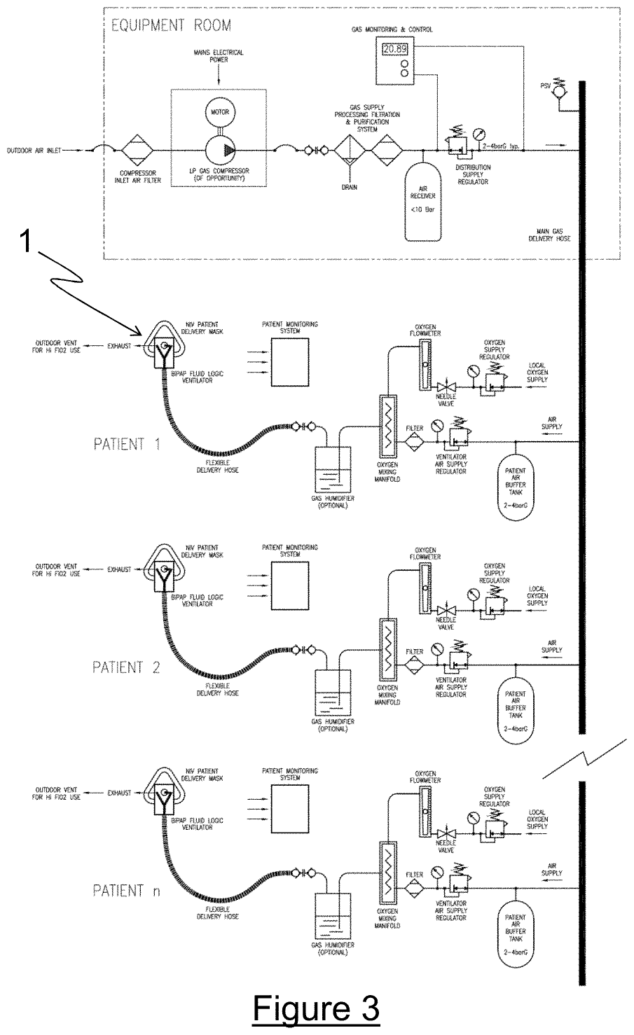

FIG. 3 shows a schematic of a ventilator system using a ventilator; and

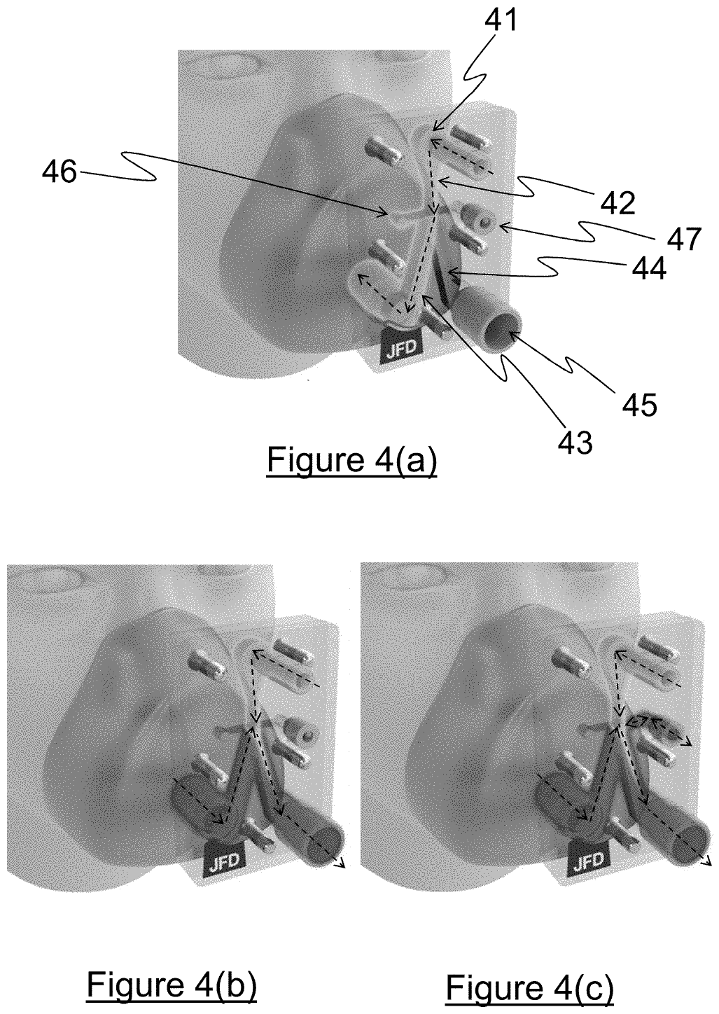

FIG. 4 shows three stages of a ventilator manifold operation.

Referring firstly to FIG. 1, there is shown a ventilator 1 secured to a schematic model patient's face 10. The ventilator 1 includes a mask 12 and a ventilator manifold 13. The manifold 13 includes an exhaust 11 and is fluidly coupled, via a fluid conduit 14, to a source of breathing gas. For the avoidance of doubt, the ventilator manifold 13 includes the closed space, openings which allow fluids to enter and leave, and surrounding material that defines the closed space and openings. There may be further components placed between the ventilator manifold 13 and the mask 12. These further components are not shown in FIG. 1.

The breathing gas may be compressed and include, for example, air, oxygen or compressed oxygen and air supplied from a mixing manifold (not shown). The mixing manifold (not shown) may permit selection of breathing gas mixture with oxygen concentrations anywhere in the range of 21-100%. The manifold 13 presents the breathing gas for a ventilator action to the patient, for example inspiration, and for exhaust of spent gas from the patient, for example exhalation, in a breathing process. As will be appreciated, the breathing gas will have a range of composition from that substantially of nascent air about the patient to higher levels of oxygen dependent upon patient requirements.

The ventilator 1 and/or ventilator manifold 13 may be formed using additive manufacturing, moulding and/or a combination of additive and subtractive methods. It will be appreciated that the material from which the manifold is formed must be acceptable for hygiene purposes and typically must be capable of being rendered sterile in a clinical environment. The ventilator 1 and/or ventilator manifold 13 may be disposable.

Now referring to FIG. 2, the ventilator manifold 13 relies upon a fluid-logic element 2 that has no moving parts. The ventilator manifold 13 provides cyclic bi-pressure ventilation that is simple and reliable. The fluid-logic element 2 uses the breathing gas to act as a valve switch.

A number of manifolds 13 may be provided (small, medium, large and the like) specifically or notionally related to patient lung capacity or patient requirements with each manifold located or otherwise assembled with the mask or ventilator as required.

During an inspiration stage (breath in), the breathing gas passes to the patient from the source of breathing gas. The breathing gas may enter a first chamber 41 via fluid conduit 14 at a pressure above ambient. The breathing gas may then follow a source path 43 via a constriction path 42, before entering a second chamber 411. The breathing gas may then exit the second chamber 411 and flow towards the mask 12. The breathing gas is drawn into the mask 12 via its positive pressure. The breathing gas flow rate may be continuous. It will be appreciated that the size and/or configuration of the chambers and paths above may be adapted to adjust the flow rate of the breathing gas. This may be advantageous to provide different breathing gas flow rates for different patients.

In an exhalation stage, the pressure of the breathing gas within the constriction path 42 directs the expelled spent breath gas to a vent path 44 and through a vent exhaust 45. The breathing gas following the constriction path 42 may also be directed to the vent path 44.

Thus, in the inspiration stage, the breathing gas is presented to the user as a slight over-pressure to facilitate ventilator action in the patient's lungs dependent upon the constriction path 42, whilst upon exhalation due to a patient's muscle and diaphragm contraction, the spent gas exhalation is directed to the vent path against the slight gas pressure through the constriction path 42.

Accordingly, in normal operation the manifold 13 and associated ventilator system will work based upon simple patient breathing action. Thus, the inhalation stage will use the patient breathing action to cause breathing gas flow to the patient and the exhalation will act against the breathing gas pressure so that this pressure acts as a ‘switch’ to urge expelled spent gas from the patient to the exhaust vent 45.

The ventilator manifold 13 is a non-mechanical device with no moving parts. The ventilator manifold 13 attaches to a breathing circuit which is in turn connected to a breathing mask 12 worn by the patient. This provides a fluid logic ventilator. A fluid logic ventilator may rely on passing a fluid (for example, breathable gas) through a Y-shaped cavity with control orifices which toggle flow automatically between the two branches of the Y-shaped cavity in a cyclic manner. The ventilator 1 may be a pressure cycled assistor-controller ventilator consisting of a single bi-stable load switched non-moving part fluid logic element.

This simple design permits a range of manufacturing methods to be employed in the construction of the ventilator, including but not limited to machining, moulding, 3D printing and fabrication. The design is such that the system can either be configured as an assembly comprising a series of individual components, or can be manufactured as a single piece component using advanced manufacturing methods such as, but not limited to selective laser sintering, stereolithographic, fusion deposition and metal additive manufacturing methods. Such a design could optionally include an integrated fluidic logic block and top plate, and may also include integrated inlet and exhaust ports, control port connectors, and the facility to incorporate a filtration device, and/or pressure relief valve, on any or all of the inlet, inhalation, and exhaust ports.

The fluid logic ventilator in a fixed form is not generally adjustable but allows the adjustment and pre-setting of key treatment parameters allowing use by a range of patients. Normally an open path is dependent upon the configuration of an inlet breathing gas chamber, the configuration (size, width, length, straight/curved/undulating and the like) of the constriction path 42, source path 43 to the ventilator mask 12, vent path 44, and also the relative angles between these elements. The angles are relative to a manifold 13 axis typically determined by the constriction path 42 with a source angle to one side of the axis and a vent angle to the other. In a conventional ventilator manifold these angles, configurations, and orientations are fixed. A set of ventilator manifolds may be provided which are fixed for different configurations so each ventilator manifold 13 is inter-changeable in the mask 12 for different results as required for specific current patient requirements and breathing gas conditions. Different sized manifolds 13 and/or different configurations of the above parameters may be provided to control the flow of breathable gas through the ventilator 1.

Further, a peak inspiratory pressure (PIP) and hence the tidal volume (VT) varies proportionally with the ventilator supply pressure allowing convenient control of the above key parameters. The end expiratory pressure is set to atmosphere, or by a PEEP valve connected downstream of the exhaust 45, the effort required to initiate a switch from inspiration to expiration is a function of the geometry of the ventilator manifold 13 and supply pressure, it changes based on supply pressure, with higher supply pressures requiring increased effort to initiate a switch. In some cases, the expiratory pressure may be pre-set by the geometry of the fluid logic element 2 defined by the ventilator manifold 13 and remains constant if fixed over typical supply pressure ranges. Further, it is possible to modify this by employing the aforementioned variable restriction feedback loops, if present. A range of minute volumes can be delivered through the use of different ventilator devices with the same geometry but with different cavity aspect ratios in the respective inter-changeable ventilator manifolds 13 in a set provided for the ventilator 1.

An alternative to providing a set of ventilator manifolds 13 is for the angles, orientations and dimensions to be adjustable in their own right but with clear constructional complications. Nevertheless, with some designs, rather than have ventilator manifold adjustment in a ventilator by using different fixed manifold sizes, a more generic manifold may be used with a length, width and eccentricity of the constriction path, source path to the ventilator, and vent path adjusted as required. Such adjustment may be by simple expansion and contraction upon adjusters as well rotation of the angles as required upon appropriate assemblies, then fixed in the desired configuration for a desired ventilator manifold and ventilator operation. Such adjusters may be conventional slip, ratchet or screw adjusters (for example, a grub screw) but also may be provided by presenting the element (source path, vent path and/or constriction path) in shape memory material which may then be fixed by curing or other means in use.

It will also be understood that constructional features such as ledges, ribs and grooves which themselves may be adjustable into and out of the flow paths can be provided as required for adjustment from a generic manifold or to allow fine adjustments by medical staff for individual current patient requirements. It will be appreciated that such adjustments may be conducted with gloved-hands. As such, grub screw type mechanisms may be preferred.

Now referring to FIG. 3, in addition to the self-contained ventilator which may be worn by the patient, the overall ventilator system further requires a suitable supply of breathing gas to the ventilator at an adjustable pressure and with the desired concentration of oxygen.

For single patient applications in a clinical situation the breathing gas provided to the ventilator 1 can simply be a hospital ring-main or cylinder-based gas supply (air, oxygen or a mixture) reduced to a pressure between 15 cmH₂O (1.47 kPa) and 400 cmH₂O (39.22 kPa) for inspiratory pressures between 5 cmH₂O (0.49 kPa) and 40 cmH₂O (3.92 kPa).

This may be advantageous for pre-clinical patient treatment in a non-hospital setting, for example: repurposed hotels, community centres or schools. In such environments there is the opportunity to deliver ventilator gas supplies to multiple ventilators (and patients) simultaneously from simple air compressors augmented by oxygen supply cylinders local to the patient, if elevated FiO2 patient treatment is indicated.

The ventilator air delivery system may comprise an air compressor to generate the increased air supply pressure, a filtration system to assure air quality and local gas storage to ensure delivery continuity. A preferred scenario is to be able to use air compressors of a variety of types, manufacturers and specifications such that existing equipment (or available equipment) can be used.

Further, this delivery system would be typically located remotely from patients to avoid issues of equipment noise and operator attendance for maintenance and operational duties.

Fluids, including breathing gas for example, may be delivered to each patient's location through a system of low-pressure fluid delivery hoses where a supply pressure would be maintained at a suitable level to feed separate local pressure regulators (and oxygen mixing manifolds) local to each patient.

Optional gas humidification may be provided at the patient's location to improve tolerance to the ventilator and patient comfort, as can a gas exhaust system that will remove high oxygen concentration exhaust gases to an outdoor vent location.

Systems may be sized to meet an expected typical treatment centre, so capacity for between 8 and 20 patients from one system may be provided, for example—or more, if required. Multiple systems may be deployed where greater numbers are needed. Of course, these are examples of numbers and capacity may be scaled to meet requirements.

FIG. 4 provides a schematic illustration of the three basic stages of ventilator manifold operation. In the inspiration (breath in) stage shown at FIG. 4(a) a breathing gas flow is connected to a chamber 41 which leads to a constriction path 42 which then bifurcates to a source path 43 end and a vent path 44 end. The breathable gas flows as shown with dashed arrowheads. The breathing gas flow acts as a ‘logic’ switch in that the breathing gas passes along the source path 43 for subsequent parts of the ventilator. The vent exhaust 45 is not open so the breathing gas is urged by its pressure though the source path 43. In some cases, the exhaust may be open, and may be connected to a PEEP value which will only open after a threshold end expiratory pressure is reached. The breathing gas may be urged through the source path 43 because a localised vortex may be created where the airflow exits 42, and because there may be a fixed volume cavity on that side, this creates an area of low pressure which effectively pulls the inlet jet over and entrains the flow against the outer wall of the source path 43. The switch may then occur when the patient's lung pressure reaches a level that is high enough to overcome this vortex and ‘push’ the inlet jet over to the exhale path. This may in turn create another vortex at the source path 43 side of the bifurcation which may help to evacuate the patient's lungs. When PEEP pressure is reached the pressure on the patient side momentarily drops below that of the exhaust side, and this may cause the flow to switch back and the cycle to repeat.

On an exhalation stage shown in FIG. 4(b) contraction of the patient's diaphragm and muscles returns spent gas from the lungs which is then directed to the vent path 44 via the source path 43 against the effective closure of the constriction path 42 by the inlet breathing gas pressure.

As will be appreciated, relative configuration, orientation and angles will determinate flow operation using the logic switch of the breathing gas pressure in the constriction path 42. Further control can be provided by regulator or control elements 46, 47 associated with the constriction path 42. One or more regulator element(s) 46, 47 may be included, in particular, two regulator elements may be included. Further regulator elements 46, 47 may be included as required.

The regulator or control elements 46, 47 are typically a path with a void at the end away from the constriction. The voids provide controlling features in terms of the breathing process and also provide sizing so that couplings to means for pressure regulation in the element 46, 47 can be provided. This pressure regulation may be with compressed air into the elements or induction of reduced pressure to stimulate flow and so assist the breathing process.

The regulator elements 46, 47 as indicated previously are normally fixed but could be adjustable in terms of length, orientation, as well as being switchable into or out of action with an operable valve, and having variable restrictions linked to either atmosphere, or a feedback loop. The regulator elements 46, 47 may be configured in a variety of ways to influence the operating parameters of the ventilator, and may act to exert a force on the inlet flow through the constriction path 42, applied via means of pressure, flow, or acoustic resonance, to influence the direction of the inlet (source) flow as it exits the constriction path 42, and direct it towards either the source path 43 or vent path 44.

Further, the regulator elements 46,47 may direct fluid flow exiting the constriction path 42 and/or source path 43. The regulator elements 46, 47 may be fluidly connected to a pressurised source. A differential pressure in a regulator element 46, 47 may provide means for directing a flow following the constriction path 42 and/or source path 43. Application of a differential pressure to either regulator element 46, 47 may provide switching of a flow path within the ventilator manifold 13. Switching frequency may be synchronised to a desired patient's requirements.

As shown in FIG. 4(c), the regulator element 47 may optionally be configured to facilitate the exhalation stage by adjustment or removal of exhaust back pressure so the exhalation through the vent path 44 to the vent exhaust 45 is provided.

Further optionally, control or regulator paths and voids may be provided in the manifold 13. These paths and voids may be actively switched into operation by applying gas pressure or a reduced pressure in timed sequence and/or in response to pressure sensors to stimulate or initiate gas flows in the inhalation stage and exhalation stage. These control or regulator paths will add some complication but will provide some ‘power-assistance’ to some patients whose breathing action is mildly compromised ensuring adequate breathing gas enters the lungs. It will also be understood that normally as can be seen in the graph shown in FIG. 2 that the inhalation stage is quicker than the exhalation stage, and this may be achieved by the use of even passive control or regulator paths and voids in terms of relative sizes and/or configuration in absolute and relative terms within a manifold.

It will be appreciated that the breathing process is one of push and pull with the breathing gas pressure being the push into the lungs with patient muscular action, and the push provided by contraction of the lungs as well as configuration of the manifold for venting of the expelled spent gas from the lungs.

It will be further appreciated that any regulator element 46, 47 may amplify a pressure and/or flow rate as required. As such, the ventilator 1 is bi-stable when oscillating and/or amplifying.

The ventilator 1 may optionally include variably controlled feedback systems on either of, or both of, the inhalation 14 and/or exhalation branches 11 providing feedback to either of, or both of the regulator elements 46, 47 to permit the flow characteristic of the circuit to be modified.

While the invention has been illustrated and described in detail in the drawings and preceding description, such illustration and description are to be considered illustrative or exemplary and non-limiting.

Other variations of the invention can be understood and effected by those skilled in the art in practicing the claimed invention, from a study of the drawings, the disclosure, and the appended claims. Each feature of the invention may be replaced by alternative features serving the same, equivalent or similar purpose, unless stated otherwise. Therefore, unless stated otherwise, each feature disclosed is one example of a generic series of equivalent or similar features.

In the claims, the word “comprising” does not exclude other elements or steps, and the indefinite article “a” or “an” does not exclude a plurality.

Any reference signs in the claims should not be construed as limiting the scope.

1. A ventilation manifold for a ventilator, the manifold comprising a fluid flow path, the flow path comprising:
bifurcated end paths, a constriction path and a compressed breathable gas chamber fluidly coupled to a compressed breathable gas source, the manifold having a manifold axis defined by the constriction path;
a source path of the bifurcated ends coupled to an outlet conduit for a ventilator to present the compressed breathable gas at a source angle to one side of the manifold axis; and
a vent path at a vent angle to the manifold axis for an exhaust flow,
the vent path acting to provide a fluid pressure valve upon an inlet compressed breathable gas from the constriction path to urge flow towards the source path and a by-pass for returned outlet spent gas flow from the source path against the inlet compressed breathable gas at the constriction path, the fluid pressure valve and the by-pass dependent upon the relative magnitudes of the source angle and the vent angle to the manifold axis and/or each other along with the configuration of the constriction path and/or the configuration of the source path and/or the vent path.

2. The ventilation manifold of claim 1, wherein the constriction path and respective source path and respective vent path are substantially symmetrical about the manifold axis to form a Y configuration.

3. The ventilation manifold of claim 1 or 2, wherein the at least one or respective source path and/or vent paths having a fixed open configuration.

4. The ventilation manifold as claimed in claim 1 or claim 2 wherein the respective source path and/or vent path are adjustable by path adjustment means.

5. A ventilation manifold as claimed in claim 4 wherein the path adjustment means is capable of varying the respective source angle and/or the vent angle in the plane of the constriction path and/or above and below the constriction path.

6. A ventilation manifold as claimed in claim 4 or claim 5 wherein the path adjustment means is capable of varying the length of the source path and/or the vent path independently or relative to each other.

7. A ventilation manifold as claimed in any of claims 4 to 6 wherein the path adjustment means is capable of variation between straight, bowed, oscillating or saw tooth configurations for the source path and/or the vent path.

8. A ventilation manifold as claimed in any of claims 4 to 7 wherein the path adjustment means is capable of adjusting the width of the source path and/or the vent path either locally along their length or along the whole length of the path independently of the other path or relative to each other.

9. A ventilation manifold as claimed in claim 1 or claim 2 or any of claims 4 to 9 wherein the constriction path has a fixed open configuration.

10. A ventilation manifold as claimed in claim 1 or claim 2 or any of claims 4 to 9 wherein the constriction path is adjustable by constriction adjustment means.

11. A ventilation manifold as claimed in claim 10 wherein the constriction adjustment means is capable of varying the orientation of the constriction path so the respective source angle and/or the vent angle in the plane of the constriction path and/or above and below the constriction path.

12. A ventilation manifold as claimed in claim 10 or claim 11 wherein the constriction adjustment means is capable of varying the length of the constriction path independently or relative to the source path and/or the vent path.

13. A ventilation manifold as claimed in any of claims 10 to 12 wherein the path adjustment means is capable of variation between straight, bowed, oscillating and saw tooth configurations for the source path and/or the vent path.

14. A ventilation manifold as claimed in any of claims 10 to 13 wherein the constriction path adjustment means is arranged to for adjusting the width of the constriction path locally or substantially along the whole length of the constriction path.

15. A ventilation manifold as claimed in any preceding claim wherein the compressed breathable gas chamber has a divergent drop cross-section narrowing to the constriction path and broader towards a connection to the connector for the compressed breathable gas.

16. A ventilation manifold as claimed in claim 15 wherein the volume of the chamber is variable by variation means.

17. A ventilation manifold as claimed in claim 16 wherein the variation means acts by adjustment in side walls of the chamber.

18. A ventilation manifold as claimed in claim 17 wherein the adjustment of the side walls is by a folded or concertina in nature for the walls.

19. A ventilation manifold as claimed in claims 16 to 18 wherein the variation means being manual with a lock to a desired volume.

20. A ventilation manifold as claimed in any of claims 16 to 18 wherein the variation means being capable of auto variation in that the volume varies with the strength of the inlet breathable gas flow to the chamber and/or the outlet gas flow through the constriction path.

21. A ventilation manifold as claimed in claim 20 wherein the auto variation has a settable pre-determined range of variation fixed by restraints upon the chamber.

22. A ventilation manifold as claimed in any preceding claim wherein the constriction path has regulator paths and/or voids to limit pressure of the inlet breathable gas flow and/or the outlet spent gas flow.

23. A ventilation manifold as claimed in claim 22 wherein the regulator paths and/or voids are selectively coupled to the constriction path by a switch means.

24. A ventilation manifold as claimed in claim 22 or claim 23 wherein the regulator paths and/or the voids being variable in terms of length and/or width and/or volume.

25. A ventilation manifold as claimed in any preceding claim wherein the ventilation manifold is part of a set of ventilation manifolds for a ventilation system whereby the flow path in the ventilation manifold of the ventilation system is always open without any mechanical valve closure and/or any mechanically operated regulation of the flow path itself, each ventilation manifold having a desired configuration for inlet compressed breathable gas flow to the source path for a ventilator and a by-pass gas flow for an outlet spent gas flow from the vent path.

26. A ventilation manifold as claimed in claim 25 wherein each ventilation manifold in the set is inter-changeable within the ventilation system.

27. A ventilation manifold as claimed in claim 25 or claim 26 wherein the ventilation manifold is configured for association with a mask having the connector for a compressed breathable gas source, the source path to a ventilator and means for exhausting the spent gas from the mask.

28. A ventilation manifold with a Y shaped flow path with bifurcated ends, a first bifurcation end as a source path for connection to a ventilator to provide compressed air and oxygen breathable gas and a second bifurcation to act as a vent with both bifurcations coupled at one end to a flow constriction path and the other end of the constriction path coupled to a compressed breathable gas supply, the configuration of the bifurcations and/or the constriction path determined to provide fluid flow direction of the compressed air and oxygen as inlet flow to first bifurcation as the source path and upon an outflow in the opposite direction of spent gas from the source path in turn to the second bifurcation against the compressed gas in the constriction path.

29. A ventilator system for ventilation of a patient comprising: a ventilation manifold as claimed in any preceding claim, wherein the source path is connected to a mixing manifold coupled to a regulated source of pressurised oxygen and a regulated source of pressurised air.

30. A system as claimed in claim 29 wherein the regulated source of pressurised oxygen is provided locally to the mixing manifold, individually or as a defined group of mixing manifolds, whilst the regulated source of pressurised air is provided generically by a compressor to a main delivery path to a plurality of mixing manifolds or each group of mixing manifolds.

31. A system as claimed in claim 30 wherein the regulated source of pressurised oxygen is a canister or bottle or tank regulated for a specific mixing manifold or a group of mixing manifolds.

32. A system as claimed in claim 30 or claim 31 wherein the mixing manifold or each group of manifolds is configured for a specific oxygen/air composition.

33. A system as claimed in claim 32 wherein there is a range of oxygen/air compositions being from about 21% environment/normal air to around 100% oxygen.

34. A system as claimed in any of claims 29 to 33 wherein the system provides mixing manifolds or groups of mixing manifolds with a known oxygen/air composition and each ventilation manifold having a connection to allow coupling one of the manifolds with its composition as required.

35. A system as claimed in any of claims 29 to 34 wherein the mixing manifolds are coupled to control means whereby the respective oxygen/air composition is maintained relative to the demand of the ventilation manifolds by adjusting the delivery of compressed oxygen by its regulated source and/or adjustment of the pressurised air by its regulated source.

36. A system as claimed in any of claims 29 to 34 wherein the system has a number of ventilation manifolds with a respective ventilation manifolds used to maintain oxygen/air composition to the breathe manifold dependent upon the demand of all the ventilation manifolds and the capability of delivery of the compressed oxygen by its regulated source and/or adjustment of the pressurised air by its regulated source.

37. A system as claimed in any of claims 29 to 36 wherein the ventilation manifold or each ventilation manifolds are coupled to at least one of the mixing manifolds by a flexible hose.

38. A system as claimed in any of claims 29 to 37 wherein the ventilation manifold is coupled to a respective ventilation manifold via a gas humidifier.

39. A kit, comprising the ventilation manifold of any preceding claim and a mask for a patient.

40. A ventilator including a ventilator manifold as claimed in any of claims 1 to 28.

41. A ventilator as claimed in claim 40 wherein the ventilator is configured to receive one of a plurality of ventilator manifolds forming a group of ventilator manifolds suitable for different breathable gas flow rates and/or for different breathable gas compositions.

42. A ventilator as claimed in claim 41 wherein each ventilator manifold of the group of ventilator manifolds is identifiably distinct with a marking such as small, medium and large or colour for a specific or notional expectation of lung capacity of patient and/or breathable gas composition.
